(19) United States
(12) Patent Application Publication
(10) Pub. No.: US 2023/0383361 A1
(43) Pub. Date: Nov. 30, 2023

(54) METHODS TO PREDICT OUTCOMES TO CHIMERIC ANTIGEN RECEPTOR T-CELLS IN LYMPHOMA FROM CELL-FREE DNA AND GENETIC MUTATIONS

(71) Applicant: The Board of Trustees of the Leland Stanford Junior University, Stanford, CA (US)

(72) Inventors: David M. KURTZ, Stanford, CA (US); Brian SWORDER, Stanford, CA (US); Arash Ash ALIZADEH, Stanford, CA (US); Maximilian DIEHN, Stanford, CA (US); Matthew FRANK, Stanford, CA (US); David B. MIKLOS, Stanford, CA (US); Crystal MACKALL, Stanford, CA (US)

(21) Appl. No.: 18/248,757

(22) PCT Filed: Oct. 12, 2021
(86) PCT No.: PCT/US21/54594

§ 371 Date: Apr. 12, 2023

Related U.S. Application Data
(60) Provisional application No. 63/091,159, filed on Oct. 13, 2020.

(51) Int. Cl. C12Q 1/6886; C12Q 1/6881

(52) U.S. Cl. CPC C12Q 1/6886; C12Q 1/6881; C12Q 2600/106; C12Q 2600/156; C12Q 2600/158

(57) ABSTRACT

The present disclosure generally relates to methods that utilize cell-free DNA from a liquid biopsy of an individual to track DNA from both the tumor and the chimeric antigen receptor (CAR) T-cells. The present disclosure further relates to methods of predicting individuals' response to therapy, e.g., CAR T-cell therapies. Additionally, the present disclosure relates to methods of treating individuals with cancer, such as lymphoma.

### CROSS-REFERENCE TO RELATED APPLICATIONS

This application claims priority to U.S. Provisional Patent Application No. 63/091,159, filed Oct. 13, 2020, the disclosure of which is incorporated by reference herein in its entirety, including any drawings.

### STATEMENT REGARDING FEDERALLY-SPONSORED RESEARCH AND DEVELOPMENT

This invention was made with U.S. government support under contract Nos. CA241076-01 and CA233975-01A1, awarded by National Cancer Institute. The U.S. Government has certain rights in this disclosure.

### BACKGROUND OF THE DISCLOSURE

Cancer is a process characterized by dramatic heterogeneity between individuals. This variation can lead to substantially different outcomes between individuals nominally sharing the same disease. For example, systemic therapy combining Rituximab with chemotherapy will cure a majority of individuals with DLBCL; however, a significant minority will still succumb to this disease. Molecular and genomic techniques have unraveled a substantial portion of this heterogeneity in DLBCL, including identification of differing molecular cell-of-origin (COO) and genomic subtypes of DLBCL.

However, most of these tools require invasive tissue biopsies to perform DNA sequencing or RNA gene expression profiling (GEP), limiting their utility. Furthermore, tissue-based classification of lymphomas and other cancers does not allow dynamic assessment of a tumor over time, including during the selective pressure of cancer therapy. Due to these limitations, methods to assess circulating tumor-derived DNA (ctDNA) from the blood plasma—or so-called ‘liquid biopsies’— have become an important tool for studying cancer genomics and tumor heterogeneity in many cancers including lymphomas. Detection of ctDNA offers a number of advantages over traditional biomarkers, including high specificity and an ability to reveal tumor-specific alterations that drive the underlying tumor. Furthermore, ctDNA allows unique opportunities for repeated assessment, providing phenotypic information demonstrating response to therapy.

Diffuse large B-cell lymphoma (DLBCL) is the most common non-Hodgkin lymphoma (NHL) and hematologic malignancy in adults. While outcomes for DLBCL have improved since the advent of Rituximab, over a third of individuals will still die from their disease. Recently, immunotherapy with engineered anti-CD19 chimeric antigen receptor (CAR19) T-cells has emerged as a highly active salvage treatment option for individuals with relapsed or refractory DLBCL. However, despite clinical activity in many individuals, over 60% of individuals treated with CAR19 T-cells will experience progressive disease and disease-related mortality. Unfortunately, the mechanisms of resistance to CAR T-cells in DLBCL remain poorly understood, with no predictive biomarkers for patient selection.

Chimeric antigen receptor (CAR) T-cells targeting CD19 (or CAR19 T-cells) are an emerging, active therapy for individuals with lymphomas. Despite high response rates to therapy, most individuals will ultimately have disease progression after CAR19 T-cells. Identifying individuals who will have favorable outcomes, versus unfavorable outcomes, after CAR19 T-cell therapies, remains a critical challenge. The disclosures provided herein provide solutions to these challenges and provide additional benefits as well.

Various references and publications may be cited throughout this specification. These publications are hereby incorporated by reference in their entirety.

### BRIEF SUMMARY OF THE DISCLOSURE

In some embodiments, the present disclosure provides a method of detection. In certain embodiments, the method comprises: a) contacting a biological sample from an individual with an agent capable of specific binding to one or more genes comprising PAX5, BTG2 and/or IRF8 genes; and b) quantitating the binding to determine the somatic mutation frequency of one or more genes comprising PAX5, BTG2 and/or IRF8 genes.

In other embodiments, the present disclosure provides a method for treating an individual having or suspected of having lymphoma, comprising administering CAR T-cell therapy to the individual with a low somatic mutation frequency in one or more genes comprising PAX5, BTG2, and IRF8. In some embodiments, the method for treating further comprises: a) contacting a biological sample from the individual with an agent capable of specific binding to one or more genes comprising PAX5, BTG2, and IRF8, b) detecting somatic mutations in the one or more genes, and c) calculating the somatic mutation frequency of the one or more genes.

In additional embodiments, the present disclosure provides a method for identifying T-cell repertoire in an individual, the method comprising: a) deep sequencing a biological sample comprising cell-free DNA (cfDNA) from the individual, b) mapping sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes.

Additionally, the present disclosure encompasses a method of identifying TCR clonotypes from cell-free DNA in an individual having or suspected of having lymphoma, the method comprising: a) deep sequencing the cell-free DNA (cfDNA) from the individual, b) mapping the sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes.

Furthermore, the present disclosure encompasses a method for monitoring therapeutic response(s) to CAR T-cell therapy in an individual having lymphoma, the method comprising: a) identifying T-cell repertoire in the individual before and/or after CAR T-cell therapy using the method for identifying T-cell repertoire in an individual described herein, and b) comparing the T-cell repertoire before and after CAR T-cell therapy, and/or at different time points after CAR T-cell therapy.

In another embodiment, the present disclosure provides a method to simultaneously track 1) tumor DNA mutations, 2) CAR T-cell DNA, and 3) T-cell clonotypes from both endogenous and engineered T-cells, or any combination of the above, in a biological sample. In certain embodiments, the method comprises: a) deep sequencing a biological sample comprising cell-free DNA (cfDNA) from the individual, b) mapping the sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes.

### BRIEF DESCRIPTION OF THE DRAWINGS/FIGURES

FIG. 1: FIG. 1A: Number of unique T-cell clonotypes (per 10³ genome equivalents) recovered by various methods of cfDNA analysis. Horizontal bar depicts the median and the box spans the interquartile range. FIG. 1B: Comparison of molecular disease burden as detected by SABER in plasma cfDNA versus PBMCs. Undetected cases (ND) are assigned a value of 1 to allow plotting on logarithmic axis. FIG. 1C: Repertoire size (total T-clonotypes per mL plasma) over time after CAR19 T-cell therapy, stratified by patient response status at the time of analysis. Error bars depict the 95% confidence interval. FIG. 1D: Spearman correlation between repertoire size one-week post-treatment and cellular CAR19 levels by FACS as well as axi-cel retroviral cfDNA CAR19 levels as measured by VirCAPP-Seq. cfDNA: cell-free DNA; G.E.: genome equivalents.

FIG. 2: CAPP-Seq for CAR19 T-cells. FIG. 2A: An overview of CAPP-Seq for tracking tumor, CAR T-cell, and TCR data from cfDNA in a single assay. FIG. 2B: Effect of pretreatment ctDNA levels on PFS during axi-cel. Kaplan Meier analysis of PFS, with individuals stratified based on pre-Axi-cel therapy ctDNA level, above and below a previously established threshold (2.5 log₁₀[haploid Genome Equivalents/mL]). FIG. 2C: Effect of molecular response by ctDNA on PFS during axi-cel therapy. A Kaplan Meier plot depicting PFS stratification for individuals with detectable versus undetectable ctDNA at day 28 after axi-cel infusion. FIG. 2D: correlation of CAR19 enumeration with CAPP-Seq vs flow cytometry. FIG. 2E: Forrest plot for effect of ctDNA and cell-free CAR DNA on PFS.

FIG. 3: Genomic determinants of CAR19 resistance. FIG. 3A: Left: Recurrently mutated genes in individuals receiving axi-cel therapy, stratified by durable remission vs. disease progression. Right: effect of mutations in given gene on PFS (hazard ratio from proportional hazard model); significant values (P<0.05) shown in green. FIG. 3B: Genes with recurrent emergent novel mutations at time of relapse after CAR19 T-cells. FIG. 3C: Clonal selection of mutations in specific genes in individuals experiencing relapse shown as a volcano plot. Mutated genes under significant positive selection are shown on the right in red; size of dot proportional to number of mutations (also shown in parentheses).

FIG. 4: PAX5 mutations in CAR19 therapy. FIG. 4A: The distribution of mutations in the PAX5 gene and enhancer locus in individuals with durable remissions vs disease progression. Top left: zoom-in on 5′ end of PAX5 gene. Top right: zoom-in on PAX5 enhancer. FIG. 4B: Location of PAX5 mutations in individuals with durable remissions vs disease progression. FIG. 4C: CD19 IHC expression in PAX5 mutated and unmutated cases from tumor biopsies at relapse. FIG. 4D Expansion and persistence of CAR19 T-cell cell-free DNA in PAX5 mutated and wild-type cases.

### DETAILED DESCRIPTION OF THE DISCLOSURE

The disclosures herein provide, in one aspect, methods to utilize cell-free DNA from a biological sample and/or liquid biopsy, such as blood plasma, of an individual to track DNA from both the tumor and the CAR T-cells themselves. By tracking both the patient's tumor and CAR19-modified T-cells, one of skill in the art can identify individuals who are likely to respond and/or not respond to therapy. Additionally, new mutations in key genes which can be predictive for poor outcomes to CAR19 therapy are identified and can be used for diagnostic and/or therapeutic purposes. These genes, including PAX5, BTG2, and IRF8, are key transcriptional regulators that define B-cell identify and control CD19 expression. In some aspects, increased mutation frequency or the presence of mutation(s) in any of the genes disclosed herein can be predictive for poor outcomes to CAR19 therapy.

The disclosure provided herein has at least two key uses: 1) tracking CAR T-cell therapeutic cells in individuals who have received this treatment. This would allow companies focused on developing CAR therapies to better monitor their individuals; 2) identifying individuals who are likely to do well with CAR19 T-cells, versus patient who are unlikely to do well. As an example, based on the inventors' research, individuals with certain mutations in key areas of the gene PAX5 are unlikely to have a favorable response to CAR19 T-cell therapy. One of skill in the art can use this biomarker for selection of an individual (e.g. patient) for their drug. To date, there is no available method to track engineered T-cell DNA from the blood plasma. There is also no available DNA-sequencing method to identify specific individuals at risk for treatment failure after CAR19 T-cell therapy.

In some embodiments, the present disclosure provides a method of detection. In some embodiments, the method comprises: a) contacting a biological sample from an individual with an agent capable of specific binding to one or more genes comprising PAX5, BTG2 and/or IRF8 genes; and b) quantitating the binding to determine the somatic mutation frequency of one or more genes comprising PAX5, BTG2 and/or IRF8 genes.

Somatic mutation can be achieved by various methods, such as but not limited to, CAPP-Seq, hybrid-capture based targeted sequencing, amplicon-based targeted sequencing, quantitative PCR, and digital PCR and other techniques readily available to one of skill in the art.

The individual can have or could be suspected of having B-cell lymphoma or B-cell leukemia. The biological sample can include, but is not limited to, circulating tumor-derived DNA (ctDNA), DNA from a tumor tissue sample, or other source of tumor DNA.

In some embodiments, the detection of somatic mutation frequency of PAX5, BTG2 and/or IRF8 genes is done simultaneously with measuring endogenous and engineered CAR T-cells.

The present disclosure further provides a method for treating an individual having or suspected of having lymphoma by administering CAR T-cell therapy to the individual with a low somatic mutation frequency in one or more genes comprising PAX5, BTG2, and IRF8. In some embodiments, the method for treating the individual further comprises: a) contacting a biological sample from the individual with an agent capable of specific binding to one or more genes comprising PAX5, BTG2, and IRF8, b) detecting somatic mutations in the one or more genes, and c) calculating the somatic mutation frequency of the one or more genes. In some embodiments, the somatic mutations can be detected by CAPP-Seq, hybrid-capture based targeted sequencing, amplicon-based targeted sequencing, quantitative PCR, and digital PCR. In other embodiments, the method further comprises comparing the mutation frequency of the one or more genes to a reference mutation frequency.

In additional embodiments, additional genes further that can be used for biomarker purposes include, but is not limited to, ZFP36L1, WHSC1, CD40, HIST1HIC, NOTCH2, TP53, and/or CD19.

In some embodiments, the individual has no mutations in one or more genes comprising PAX5, BTG2, and IRF8. In other embodiments, the individual has a mutation frequency no greater than 0.05% allele fraction in one or more genes comprising PAX5, BTG2, and IRF8. In certain embodiments, the individual without mutation or with a mutation frequency no greater than 0.05% allele fraction in one or more genes comprising PAX5, BTG2, and IRF8 has favorable response to CAR T-cell therapy. In some specific embodiments, the individual without mutation or with a mutation frequency no greater than 0.05% allele fraction in the enhancer and 5′ UTR regions of PAX5 has favorable response to CAR T-cell therapy.

In some embodiments, the CAR T-cell therapy comprises a CAR19 therapy. In some embodiments, the CAR T-cell therapy comprises Axicabtagene ciloleucel.

The present disclosure also provides methods for identifying T-cell repertoire in an individual, the method comprising: a) deep sequencing a biological sample comprising cell-free DNA (cfDNA) from the individual, b) mapping sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes. Such methods can be achieved by non-limiting protocols and procedures as shown in the Examples.

For all of the methods described herein, the biological sample can be a bodily fluid, such as, but not limited to, plasma, urine, and cerebrospinal fluid.

In some embodiments, the individual has or is suspected of having a cancer. In certain embodiments, the cancer is lymphoma. In some embodiments, the individual has had CAR T-cell therapy. In some exemplary embodiments, the CAR T-cell therapy is axicabtagene ciloleucel.

In further embodiments, the present disclosure encompasses a method for identifying T-cell repertoire in an individual, the method comprising: a) deep sequencing a biological sample comprising cell-free DNA (cfDNA) from the individual, b) mapping sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes. Such methods can be achieved by non-limiting protocols and procedures as shown in the Examples.

Additionally, the present disclosure encompasses a method of identifying TCR clonotypes from cell-free DNA in an individual having or suspected of having lymphoma, the method comprising: a) deep sequencing the cell-free DNA (cfDNA) from the individual, b) mapping the sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes. Such methods can be achieved by non-limiting protocols and procedures as shown in the Examples.

Furthermore, the present disclosure encompasses a method for monitoring therapeutic response(s) to CAR T-cell therapy in an individual having lymphoma, the method comprising: a) identifying T-cell repertoire in the individual before and/or after CAR T-cell therapy using the method for identifying T-cell repertoire in an individual described herein, and b) comparing the T-cell repertoire before and after CAR T-cell therapy, and/or at different time points after CAR T-cell therapy. The T-cell repertoire can be expanded after the CAR T-cell therapy. The T-cell repertoire expansion can include greater total TCR-β clonotypes. In addition, the number of total TCR-β clonotypes can correlate with a favorable response to CAR T-cell therapy. In certain embodiments, the individual has relapsed/refractory diffuse large B-cell lymphoma (rrDLBCL).

The present disclosure also provides methods to simultaneously track 1) tumor DNA mutations, 2) CAR T-cell DNA, and 3) T-cell clonotypes from both endogenous and engineered T-cells, or any combination of the above, in a biological sample. These methods can be achieved by: a) deep sequencing a biological sample comprising cell-free DNA (cfDNA) from the individual, b) mapping the sequencing reads to identify candidate rearrangements within TCR loci, c) identifying unique cfDNA fragments by resolving consensus of unique molecular identifiers (UMI) clustered by Levenshtein distances, and d) CDR3-anchoring for enumeration of final receptor clonotypes. For these methods, the biological sample can be a bodily fluid (e.g., plasma, urine, and cerebrospinal fluid).

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1: Method for Simultaneous Profiling of Circulating Tumor DNA (ctDNA), Chimeric Antigen Receptor Derived Cell-Free DNA and T-Cell Receptor Clonotypes (SABER)

Cell-free DNA (cfDNA) was extracted from plasma and sequencing libraries were prepared as previously described (Scherer et al, Science Translational Medicine 2016, Chabon et al Nature 2020). A novel hybridization capture oligonucleotide panel was designed to facilitate the simultaneous profiling of ctDNA, CAR T-cell derived cfDNA and rearranged T-cell receptor genes.

For ctDNA profiling, genomic regions known to be recurrently altered in lymphoma, genes of known functional significance in lymphoma and genes potentially involved in CAR T-cell resistance were targeted. Probes targeting these genomic regions were synthesized from Roche Nimblegen (NimbleDesign portal).

Probes targeting the CAR T-cell vector, rearranged T-cell receptor genes and genes putatively involved in CAR T-cell resistance were designed using the IDT xGen Lockdown Probe tool.

Hybrid capture (SeqCap EZ Choice, NimbleGen) was performed according to the manufacturer's protocol, with the exception that hypotaurine (Sigma-Aldrich, H1384) was added to the hybrid capture reaction at a final working concentration of 5 mM. All capture steps were conducted on a thermal cycler at 47° C. Following hybrid capture, data analysis was performed as follows.

Circulating Tumor DNA (ctDNA) Profiling

ctDNA profiling to characterize single nucleotide variants (SNVs), copy number alterations (CNAs) and insertions/deletions (indels) was performed as previously described (Scherer et al, Science Translational Medicine 2016, Chabon et al Nature 2020).

CAR T-Cell cfDNA Profiling

Quantification and profiling of CAR T-cell derived cfDNA was performed by mapping cfDNA reads to a modified human genome consisting of hg19 augmented with the CAR T-cell transduction vector reference sequence. Only reads aligned to the integrated portion of the retroviral vector were considered for analysis, and reads aligned to unintegrated vector backbone were discarded. The 75th percentile of read depth across the integrated portion of the retroviral reference genome was used to quantify CAR T-cell levels, allowing for both high sensitivity and specificity. The ratio of depth mapping to CAR T-cell loci and human loci was also considered to define the ‘fraction of CAR T-cell DNA’ in a sample. Retroviral cfDNA fragment lengths were inferred from the distance between properly mapped, paired-end reads.

TCR Receptor Profiling—SABER

SABER (Sequence Affinity capture & analysis By Enumeration of cell-free Receptors) is developed as a technique for TCR enrichment and analysis of fragmented rearrangements shed in cfDNA. TCR enrichment is achieved via Cancer Personalized Profiling by Deep Sequencing (CAPP-Seq) after which raw sequencing data is processed and quantitated into unique clonotypes. Candidate rearrangements within TCR loci are identified first by mapping sequencing reads to hg38. Within this space of potential rearrangements, PCR duplicates are resolved by a novel strategy that defines the consensus of unique molecular identifiers clustered by Levenshtein distances. Unique cfDNA fragments are probed for CDR3-containing regions by anchoring to IMGT-defined CDR3 start and end motifs. Fragments containing full-length and incomplete CDR3s are allowed through. A second filtering stage mediated by Blast (NCBI) and Blat (UCSC Genome Browser) removes putative CDR3 reads containing templated DNA, to arrive at a final repertoire of TCR-a, TCR-β, TCR-δ, and TCR-y clonotypes. SABER thus leverages information from fragmented TCRs, a critical requirement for cfDNA, to make V gene, CDR3, and J gene assignments.

### Example 2: Profiling T-Cell Receptor Diversity and Dynamics During Lymphoma Immunotherapy Using Cell-Free DNA (cfDNA)

Characterization of T-cell receptor (TCR) diversity and dynamics is increasingly critical to understanding therapeutic immune responses targeting tumors. Current TCR profiling methods generally require invasive tissue biopsies that capture a single snapshot of immune activity or are limited by the sheer diversity of the circulating TCR repertoire, estimated to vary between 1e7 and ˜1e15 clonotypes (Robins HS, 2009 Blood). In theory, T-cells with the greatest turnover could best reflect pivotal immune dynamics from both circulating and tissue-derived compartments, including non-circulating tissue-resident memory T-cells (Trm). To noninvasively capture such responses in the blood, a high-throughput TCR profiling approach is developed and benchmarked using plasma, optimized for the fragmented nature of cfDNA and the non-templated nature of rearranged TCRs. This method is applied for residual disease monitoring in mature T-cell lymphomas (TCL) without circulating disease, and for characterizing immune dynamics after anti-CD19 chimeric antigen receptor (CAR19) T-cell therapy of B-cell lymphomas with axicabtagene ciloleucel.

Sequence Affinity capture & analysis By Enumeration of cell-free Receptors (SABER) was developed as a technique for TCR enrichment and analysis of fragmented rearrangements shed in cfDNA, and applied this method using Cancer Personalized Profiling by Deep Sequencing (CAPP-Seq). SABER was used to profile a total of 381 samples (300 cfDNA and 81 PBMC samples) from 77 lymphoma individuals and 16 healthy controls. After mapping sequencing reads (hg38) to identify candidate rearrangements within TCR loci, unique cfDNA fragments were resolved by a novel strategy to define consensus of unique molecular identifiers clustered by Levenshtein distances, followed by CDR3-anchoring for enumeration of final receptor clonotypes. SABER thus leverages information from fragmented TCRs, a critical requirement for cfDNA, to make V gene, CDR3, and J gene assignments after deduplication-mediated error-correction. SABER was benchmarked against established amplicon-based TCR-β targeted sequencing (LymphoTrack, Invivoscribe) and repertoire analysis methods (MiXCR; Bolotin et al, 2015 Nature Methods) when considering both cfDNA and PBMC samples from healthy adults and TCL individuals. Malignant TCL clonotypes were identified in tumor specimens using clonoSEQ (Adaptive Biotechnologies). SABER performance was assessed for tracking clonal molecular disease in individuals with mature TCLs from both cellular and cell-free circulating compartments (n=9). Finally, TCR repertoire dynamics over time was evaluated in 66 DLBCL individuals after CAR19 T-cell therapy.

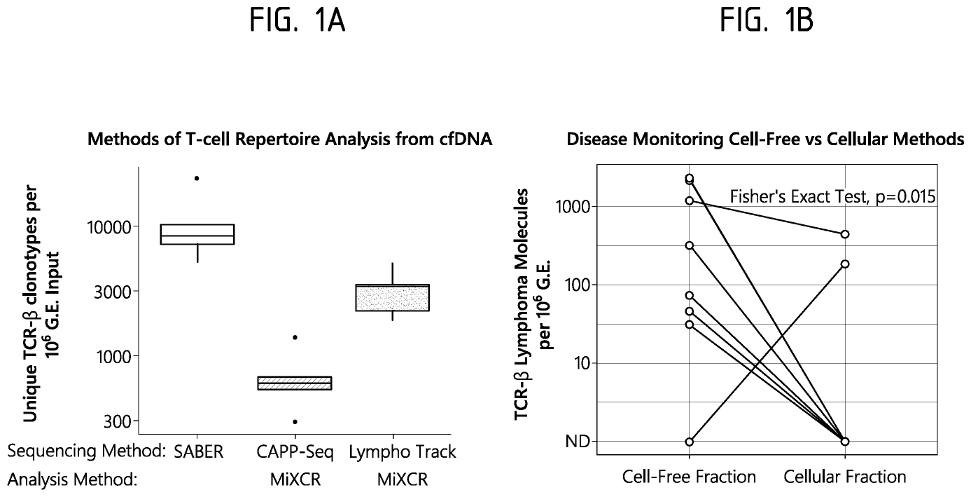

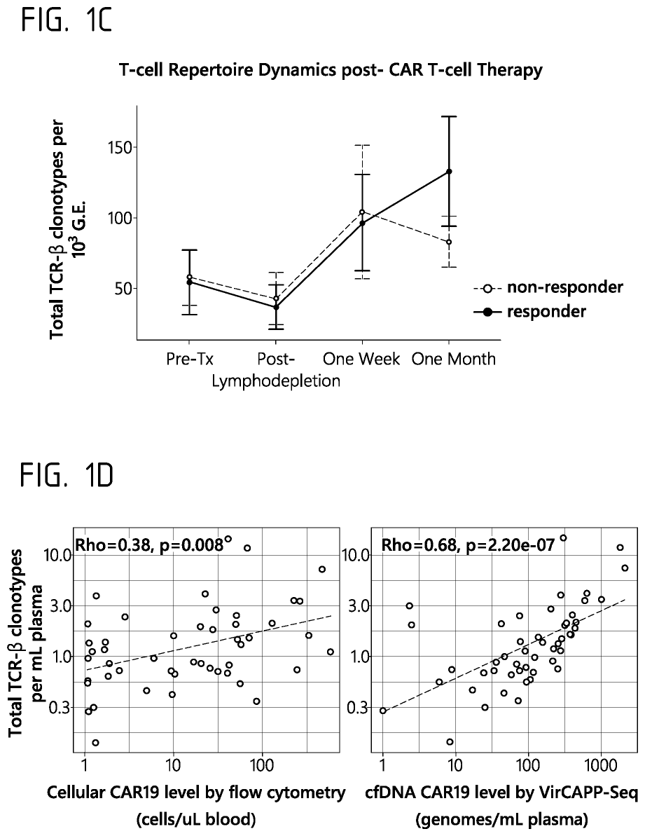

SABER demonstrated superior recovery of TCR clonotypes from cfDNA compared to both amplicon sequencing (LymphoTrack, Invivoscribe) and hybrid-capture methods when enumerating receptors using MiXCR (FIG. 1A). When applied to pre-treatment blood samples from TCL individuals, SABER identified the malignant clonal TCR-β rearrangement in 8/9 (88.9%) cases, with significantly improved detection in cfDNA (p=0.015, FIG. 1B). Specifically, tumoral TCR clonotype was detectable only in cfDNA in 6 cases (75%), cfDNA-enriched in 1 case (12.5%), and detectable only in PBMC in 1 case (12.5%). SABER was applies to monitor TCR repertoire dynamics in cfDNA after CAR T-cell therapy of individuals with relapsed/refractory DLBCL and observed increased T-cell turnover and repertoire expansion (greater total TCR-β clonotypes) (FIG. 1C). As early as 1-week after CAR19 infusion, TCR repertoire size was significantly correlated both with cellular CAR19 T-cell levels by flow cytometry (p=0.008), as well as with retroviral CAR19 levels in cfDNA (p=2.2e-07) suggesting faithful monitoring of CAR T-cell activity (FIG. 1D). TCR repertoire size 28 days after infusion was significantly associated with longer progression-free survival (HR 0.246, 95% CI 0.080-0.754, p=0.014).

SABER has a favorable profile for cfDNA TCR repertoire capture when compared to existing methods and could thus have potential broad applicability to diverse disease contexts. Given the higher abundance of lymphoma derived TCRs in cfDNA than intact circulating leukocytes, SABER holds promise for monitoring minimal residual disease in T-cell lymphomas. This approach also holds promise for monitoring T-cell repertoire changes including after CAR T-cell therapy and for predicting therapeutic responses.

### Example 3: Characterizing Genomic Mechanisms of Resistance to Engineered T-Cell Therapy in B-Cell Lymphomas

Methods to detect tumor-derived cell-free DNA from the blood plasma of individuals, or so-called circulating tumor DNA (ctDNA), have unlocked significant opportunities to study tumor biology both prior to and after therapy. Cancer Personalized Profiling by Deep Sequencing (CAPP-Seq) was previously applied, a targeted sequencing approach for ultra-sensitive detection of ctDNA, to individuals with DLBCL. In this work, CAPP-Seq was demonstrated as a robust method for disease detection, mutational genotyping, and molecular disease monitoring during first-line treatment. More recently, CAPP-Seq and novel techniques were further developed for measuring both tumor and engineered T-cell DNA to simultaneously monitor therapy and disease in individuals receiving CAR19 T-cells. By using these tools to study individuals with responsive and non-responsive disease, we have uncovered several candidate mutations potentially predicting for adverse outcomes after CAR19 therapy. These candidate resistance mechanisms largely fall into two categories: 1) alterations in CD19, the target of CAR19 therapy itself, and 2) genes that define B-cell identity, such as PAX5, IRF8, and BTG2.

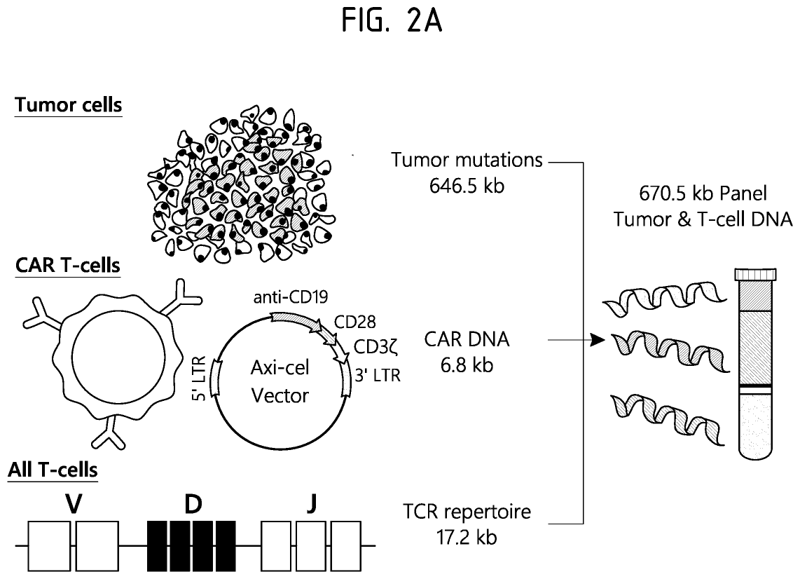

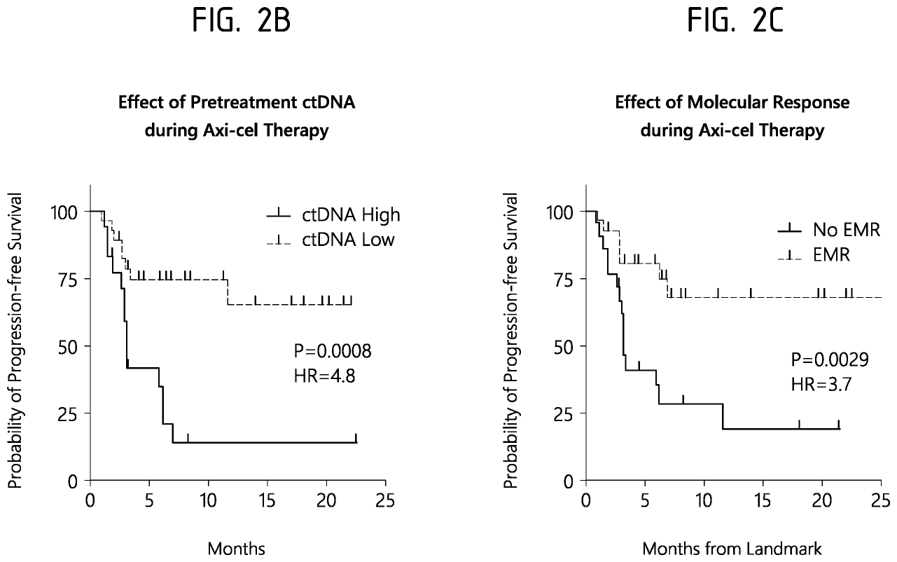

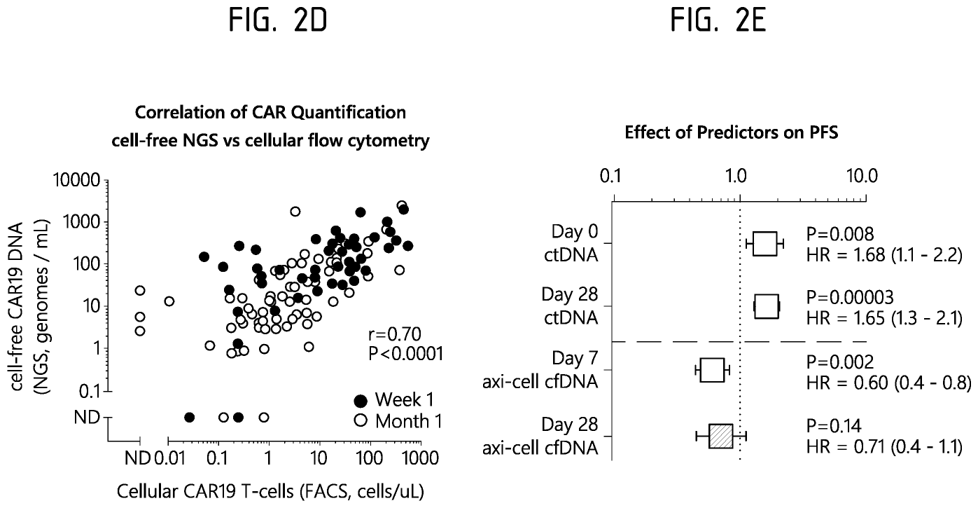

Further, we have developed an expanded CAPP-Seq panel designed to interrogate three separate sources of cfDNA 1) somatic tumor mutations, including potential resistance mechanisms to CAR19 therapy, 2) CAR19 T-cell DNA from the CAR transgene, and 3) rearranged T-cell receptors from both endogenous and engineered T-cells (FIG. 2A). We used this assay to assess ctDNA and cell-free CAR DNA from a cohort of 65 individuals with relapsed/refractory large B-cell lymphoma receiving the FDA approved therapy axicabtagene ciloleucel (axi-cel) using plasma samples prior to lymphodepletion chemotherapy, and at days 0, 7, and 28 after treatment. We also assessed samples at the time of relapse where available. Using this assay, we genotyped a median of 106 (IQR: 5.5-223) mutations per patient to enable disease quantification. Importantly, we were able to demonstrate the prognostic significance of both pretreatment ctDNA levels and an Early Molecular Response at day 28 (FIGS. 2B-2C). These findings are similar to individuals undergoing first-line therapy with R-CHOP, and suggest broad utility for ctDNA across diverse therapies. Furthermore, the performance of the assay to quantify CAR T-cells provided herein is assessed by comparing cell-fee CAR enumeration by CAPP-Seq to flow cytometry on PBMCs using fluorescently labeled antibodies against the anti-CD19 scFv (FMC63). Interestingly, CAR T-cell enumeration from flow cytometry on PBMCs and targeted NGS from cell-free DNA were highly correlated across samples (FIG. 2D). When assessed as continuous variables, as expected, higher levels of ctDNA both prior to treatment and at day +28 were associated with adverse outcomes, while higher levels of CAR-derived DNA at day +7 (the time of peak CAR expansion) was associated with improved outcomes (FIG. 2E).

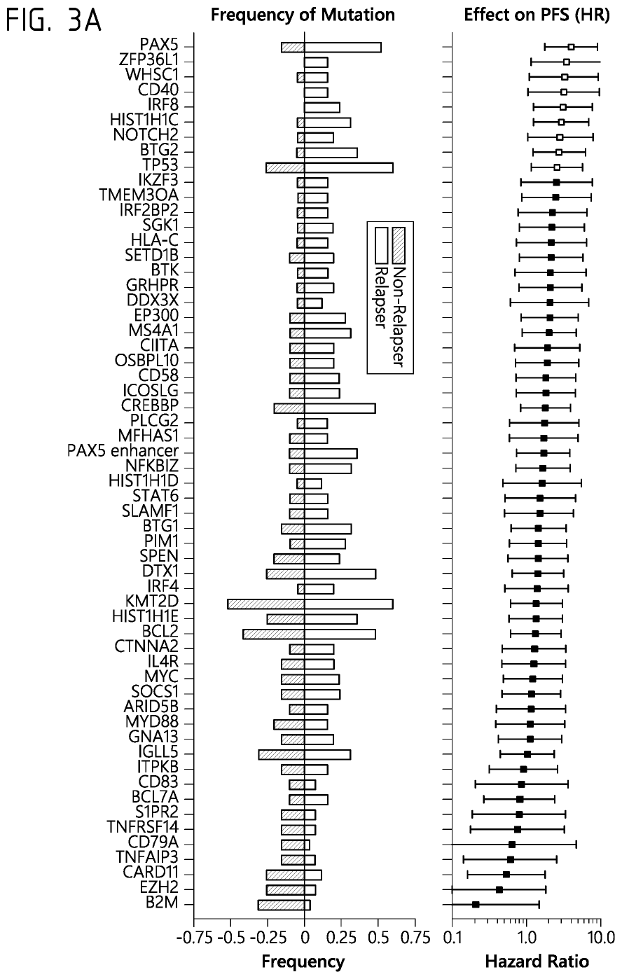

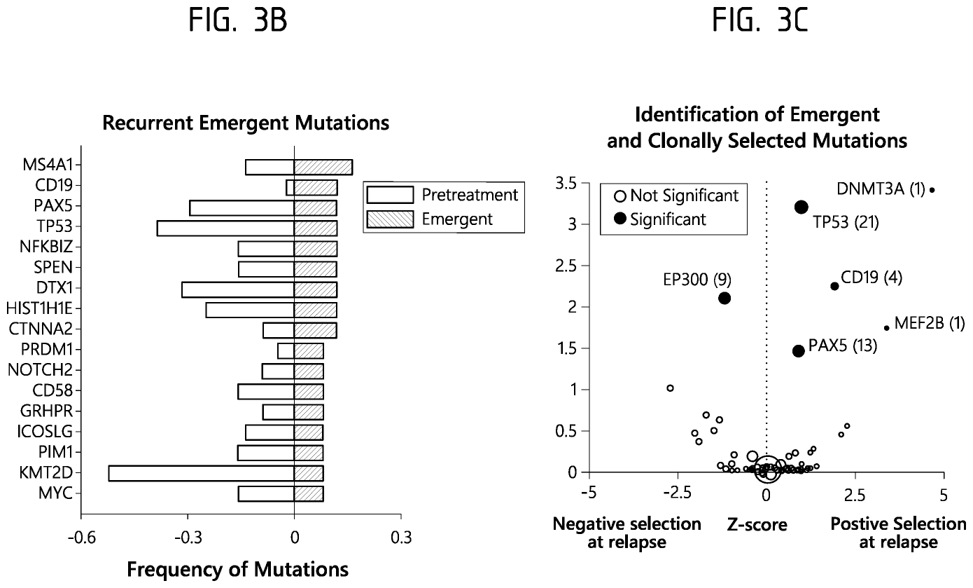

Furthermore, using samples prior to therapy and at the time of relapse, we noninvasively genotyped mutations in these cases, including SNVs and insertions and deletions (indels) in the CAPP-Seq panel, as well as performing genome-wide assessment of SCNAs. We identified recurrent genomic alterations in several candidate genes that were significantly associated with inferior outcomes (FIG. 3A). Notably, three of the genes found to have the most significant impact are known transcription factors or regulators of transcription associated with B-cell identity PAX5, IRF8 and BTG2. Moreover, when assessing individuals failing CAR19 T-cell therapy, we observed striking recurrence of emergent mutations in multiple genes. These include perhaps unsurprisingly CD19, the target of CAR19 therapy, but also PAX5 as well as TP53 (FIGS. 3B-3C).

Notably, PAX5 is a central regulator of B-cell identity that is necessary for B-cell development, and controls multiple core phenotypic characteristics of B-cells, such as CD19 expression. Further, PAX5 alterations have been implicated in the oncogenesis of multiple B-cell derived neoplasms.

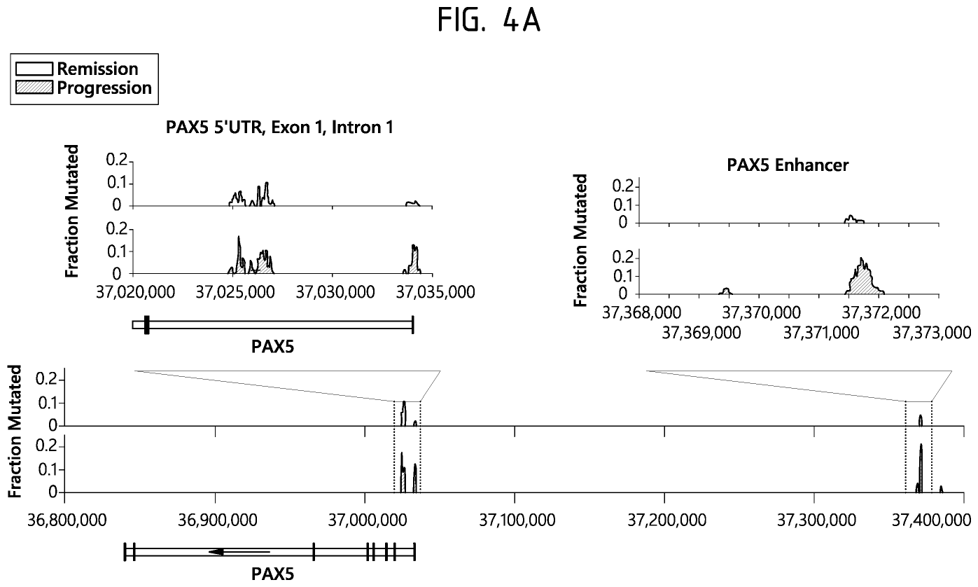

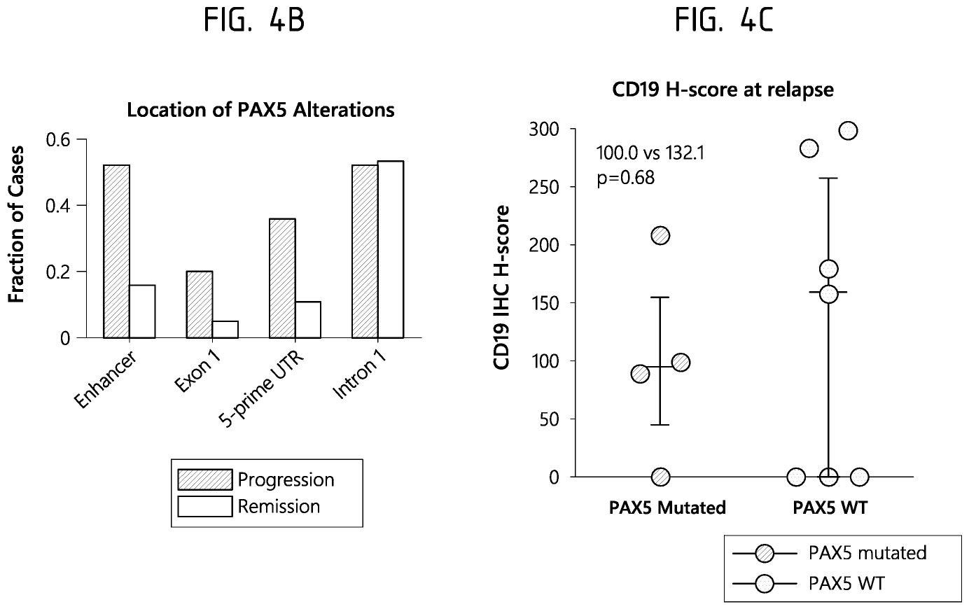

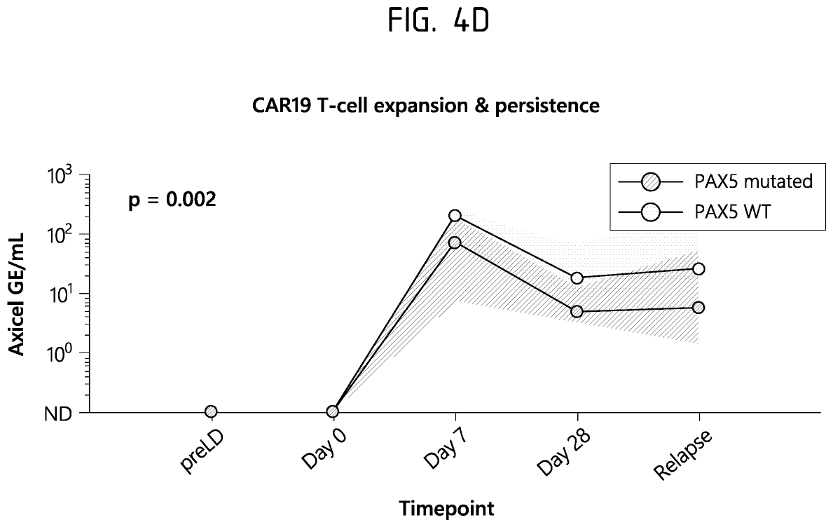

While the initial results identify PAX5 mutations as a potential candidate mechanism for resistance to CAR19 therapy, the mechanism by which these mutations act is not clear. Some insight can be drawn from the fact that most mutations in PAX5 in the cohort are not found in coding regions, but are instead found in regulatory elements including the 5′UTR and a distal enhancer region (FIGS. 4A-4B). Interestingly, this enhancer region 300 kb upstream of the gene body has been associated with regulation of PAX5 expression, where enhancer mutations in CLL have been associated with decreased expression of PAX5. Given that PAX5 is known to control CD19 expression, one possible hypothesis is that these mutations lead to “on-target” resistance mediated through CD19 loss. However, in an initial study of CD19 protein expression by IHC in the axi-cel cohort, a difference in CD19 H-score in cases with or without PAX5 alterations was not observed (FIG. 4C). Interestingly, we did observe a significant decrease in expansion of CAR19 T-cell DNA in cell-free DNA in PAX5 mutated cases, as compared with unmutated cases (FIG. 4D).

In brief summary, to date, we have used CAPP-Seq to profile ctDNA from a cohort of 65 individuals with relapsed/refractory large B-cell lymphoma receiving the FDA approved therapy axicabtagene ciloleucel (axi-cel) at Stanford University (Table 1). All individuals in this cohort have received at least 2 prior therapies, with a median follow-up of 7.16 months. To date, 48% (31/65) of individuals have relapsed after CAR19 therapy. ctDNA was isolated and sequenced using CAPP-Seq at multiple timepoints relative to CAR19 infusion in these individuals including: pre-lymphodepletion, day 0, day 7, day 28 and when available, the time of relapse.

TABLE 1
Characteristics of relapsed/refractory large B-cell lymphoma
individuals treated with axicabtagene ciloleucel (axi-cel) at
Stanford University.

| | Cohort (n = 65) | |
|---|---|---|
| Age | 59 | (21-82) |
| Gender M/F | 43/22 | |
| # Prior Therapies | | |
| 2 | 27 | (42%) |
| 3 | 17 | (26%) |
| 4 | 12 | (18%) |
| ≥5 | 6 | (9%) |
| Prior Auto SCT | 13 | (20% |
| Lymphoma Subtype | | |
| non-GCB DLBCL | 25 | (39%) |
| GCB DLBCL | 18 | (28%) |
| Transformed Follicular | 16 | (25%) |
| Primary Mediastinal | 3 | (5%) |
| Double Hit | 11 | (17%) |
| Follow-up (months) | 7.16 | (1.13-25.23) |
| Non-progressor | 34 | (52%) |
| Progressor | 31 | (48%) |

Using a previously established ctDNA threshold to stratify disease burden (2.5 log₁₀(hGE/mL)) we observed significantly superior progression-free survival (PFS) in individuals with low pretreatment ctDNA levels treated with axi-cel (FIG. 2B). Additionally, we found that interim ctDNA levels were also potentially predictive of outcomes, as individuals with detectable ctDNA levels on day 28 after CAR19 infusion had significantly inferior PFS relative to individuals with undetectable ctDNA levels at this same time point (FIG. 2C). Thus suggesting that pre-treatment ctDNA levels and molecular responses are prognostic of outcomes in relapsed/refractory large B-cell lymphoma individuals receiving CAR19 therapy. Additionally, CAPP-Seq was used to identify genomic alterations in this cohort, including: single nucleotide variants (SNVs), small insertions/deletions (indels) and somatic copy number alterations (SCNAs). We identified recurrent genomic alterations in several candidate genes that were significantly associated with inferior outcomes in these individuals. Notably, three of the genes found to have the most significant impact are the B-cell transcription factors PAX5 and IRF8, as well as the transcriptional coregulator BTG2. (FIG. 3A). Indeed, these genes were among the most differentially altered between the individuals who relapsed after CAR19 therapy versus those that did not, with 88% of relapsing individuals having an alteration in at least one of genes, compared with only 26% of non-relapsing individuals having an alteration in these same genes (FIG. 3A).

What is claimed is:

1. A method of detection comprising:
a. contacting a biological sample from an individual with an agent capable of specific binding to one or more genes comprising PAX5, BTG2 and/or IRF8 genes;
b. quantitating the binding to determine the somatic mutation frequency of one or more genes comprising PAX5, BTG2 and/or IRF8 genes.

2. The method of claim 1, wherein the individual has or is suspected of having B-cell lymphoma or B-cell leukemia.

3. The method of claim 1 or 2, wherein the biological sample comprises circulating tumor-derived DNA (ctDNA), DNA from a tumor tissue sample, or other source of tumor DNA.

4. The method of any one of the preceding claims, wherein the detection of somatic mutation frequency of PAX5, BTG2 and/or IRF8 genes is done simultaneously with measuring endogenous and engineered CAR T-cells.

5. A method for treating an individual having or suspected of having lymphoma, the method comprising administering CAR T-cell therapy to the individual with a low somatic mutation frequency in one or more genes comprising PAX5, BTG2, and IRF8.

6. The method of claim 5, further comprising:
a. contacting a biological sample from the individual with an agent capable of specific binding to one or more genes comprising PAX5, BTG2, and IRF8,
b. detecting somatic mutations in the one or more genes, and
c. calculating the somatic mutation frequency of the one or more genes.

7. The method of claim 5 or 6, wherein the somatic mutations are detected by CAPP-Seq, hybrid-capture based targeted sequencing, amplicon-based targeted sequencing, quantitative PCR, and digital PCR.

8. The method of any one of claims 5-7, wherein the method further comprises comparing the mutation frequency of the one or more genes to a reference mutation frequency.

9. The method of any one of claims 5-8, wherein the one or more genes further comprises ZFP36L1, WHSC1, CD40, HIST1HIC, NOTCH2, and TP53.

10. The method of any one of claims 5-9, wherein the one or more genes further comprises CD19.

11. The method of any one of claims 5-10, wherein the individual has no mutations in one or more genes comprising PAX5, BTG2, and IRF8.

12. The method of any one of claims 5-10, wherein the individual has a mutation frequency no greater than 0.05% allele fraction in one or more genes comprising PAX5, BTG2, and IRF8.

13. The method of any one of claims 5-12, wherein the individual without mutation or with a mutation frequency no greater than 0.05% allele fraction in one or more genes comprising PAX5, BTG2, and IRF8 has favorable response to CAR T-cell therapy.

14. The method of any one of claims 5-13, wherein the individual without mutation or with a mutation frequency no greater than 0.05% allele fraction in the enhancer and 5′ UTR regions of PAX5 has favorable response to CAR T-cell therapy.

15. The method of any one of claims 5-14, wherein the CAR T-cell therapy comprises a CAR19 therapy.

16. The method of any one of claims 5-15, wherein the CAR T-cell therapy comprises Axicabtagene ciloleucel.

17. A method for identifying T-cell repertoire in an individual, the method comprising:
a. deep sequencing a biological sample comprising cell-free DNA (cfDNA) from the individual,
b. mapping sequencing reads to identify candidate rearrangements within TCR loci,
c. identifying unique cfDNA fragments by resolving consensus of unique molecular identifiers (UMI) clustered by Levenshtein distances, and
d. CDR3-anchoring for enumeration of final receptor clonotypes.

18. The method of claim 17, wherein the biological sample is a bodily fluid.

19. The method of claim 18, wherein the bodily fluid comprises plasma, urine, and cerebrospinal fluid.

20. The method of any one of claims 17-19, wherein the individual has or is suspected of having a cancer.

21. The method of claim 20, wherein the cancer is lymphoma.

22. The method of any one of claims 17-21, wherein the individual has had CAR T-cell therapy.

23. The method of claim 22, wherein the CAR T-cell therapy is axicabtagene ciloleucel.

24. A method of identifying TCR clonotypes from cell-free DNA in an individual having or suspected of having lymphoma, the method comprising:
a. deep sequencing the cell-free DNA (cfDNA) from the individual,
b. mapping the sequencing reads to identify candidate rearrangements within TCR loci,
c. identifying unique cfDNA fragments by resolving consensus of unique molecular identifiers (UMI) clustered by Levenshtein distances, and
d. CDR3-anchoring for enumeration of final receptor clonotypes.

25. A method for monitoring therapeutic response(s) to CAR T-cell therapy in an individual having lymphoma, the method comprising:
a. identifying T-cell repertoire in the individual before and/or after CAR T-cell therapy using the method of claim 17, respectively, and
b. comparing the T-cell repertoire before and after CAR T-cell therapy, and/or at different time points after CAR T-cell therapy.

26. The method of claim 25, wherein the T-cell repertoire expands after the CAR T-cell therapy.

27. The method of claim 26, wherein the T-cell repertoire expansion includes greater total TCR-β clonotypes.

28. The method of claim 27, wherein the number of total TCR-β clonotypes correlates with the favorable response to CAR T-cell therapy.

29. The method of any one of claims 25-28, wherein the individual has relapsed/refractory diffuse large B-cell lymphoma (rrDLBCL).

30. A method to simultaneously track 1) tumor DNA mutations, 2) CAR T-cell DNA, and 3) T-cell clonotypes from both endogenous and engineered T-cells, or any combination of the above, in a biological sample, the method comprising:
a. deep sequencing a biological sample comprising cell-free DNA (cfDNA) from the individual,
b. mapping the sequencing reads to identify candidate rearrangements within TCR loci,
c. identifying unique cfDNA fragments by resolving consensus of unique molecular identifiers (UMI) clustered by Levenshtein distances, and
d. CDR3-anchoring for enumeration of final receptor clonotypes.

31. The method of claim 30, wherein the biological sample is a bodily fluid.

32. The method of claim 31, wherein the bodily fluid comprises plasma, urine, and cerebrospinal fluid.
